(19) United States
(12) Patent Application Publication
(10) Pub. No.: US 2007/0225588 A1
(43) Pub. Date: Sep. 27, 2007

(54) Automated Patient Localization in a Medical Imaging System

(76) Inventor: Michael Steckner, Richmond Heights, OH (US)

Correspondence Address: BUCKINGHAM, DOOLITTLE & BURROUGHS, LLP, 3800 EMBASSY PARKWAY, SUITE 300, AKRON, OH, 44333-8332, US

(21) Appl. No.: 11/308,350

(22) Filed: Mar. 17, 2006

(51) Int. Cl. A61B 5/05

(52) U.S. Cl. 600/407; 600/424

(57) ABSTRACT

Described herein is a process for patient localization within a medical imaging system, having a first and second signal means for identifying patient position. The patient is manually positioned on a patient table at an initial position outside the system. A first signal means is manually positioned adjacent an area of interest on the patient in the initial position and the first signal means communicates that initial patient position to a detection means. The second signal means communicates a desired final patient position location to the detection means. The detection means either essentially continuously monitors and compares said initial and subsequent positions to the final position, or calculates the distance between the initial position and the final position and causes the patient to move from the initial position to the final position when the positions are not essentially the same.

### TECHNICAL FIELD

The invention relates generally to medical imaging systems, and more specifically to the automated alignment of patients within such systems.

### BACKGROUND OF THE INVENTION

Medical imaging is the process of using a specialized system to create an image of a person's internal organs, tissues, and other structures and characteristics that cannot commonly be seen with the human eye. Some medical imaging systems are large in size and require the patient to be brought within the system for imaging. For these types of systems, commonly a patient is positioned on a movable table that can advance into the medical imaging system. The patient is positioned at a specific reference point within the system, and imaging by the system occurs.

Magnetic resonance imaging (MRI) is a commonly used medical imaging technology. Within a magnetic resonance imaging (MRI) system a patient table, commonly called the patient couch, extends into the bore of the magnet, and exists to support and position the patient so the patient can lie comfortably during the imaging process. The couch houses mechanical as well as electrical components that allow the patient and the couch to be moved into the center of the magnet bore, known as the isocenter, where the optimal imaging area of the magnet is located and imaging occurs.

FIG. 1 illustrates a system utilizing prior art. Typically, patient 12 is brought into the scan room and instructed to lie down on patient couch 14 of MRI system 10. Patient 12 is positioned on table 14 and desired anatomy 18 to be imaged is positioned within, on, under, or in some other relation to a device known as RF coil 16. The proximity of RF coil 16 to the appropriate part of the body in question is critical to the quality of the resultant image.

Cross-hair laser 20 existing on the facade of the MRI system are then turned on. Couch 14 is advanced partially into magnet 26, and RF coil 16 with anatomy of interest 18 centered inside is positioned under laser cross-hairs 20. Cross-hairs 20 of the facade laser are a pre-determined distance from isocenter 24 of magnet 26, such that when an object is centered under cross-hairs 20 and the appropriate button, which in FIG. 1 (PRIOR ART) is “Set” button 22, is pushed on MRI system 10 patient couch 14 advances the pre-set distance required to position the appropriate part of the body at magnet isocenter 24. It is necessary for the cross-hair lasers to be positioned away from the isocenter of the magnet so the technologist, or person who runs the imaging session, can conveniently and easily see the centering, as the bore of the magnet is difficult to see within to center the patient. Thus, the positioning of the patient is essentially a three-step process: center the patient in the RF coil; center the RF coil under the laser cross-hairs; and advance the couch to isocenter of the magnet.

Attempts have been made in other medical imaging procedures with similar requirements for patient positioning to expedite and automate the patient positioning process. U.S. Pat. No. 6,662,036 (issued Dec. 9, 2003) teaches of a surgical positioning system for positioning and repositioning a portion of a patient's body for medical treatment or imaging. The patented system utilizes multiple cameras to view the body and the surgical or imaging machine, which identify index markers that are located in relationship with the portion of the patient's body that is of interest during the original imaging or surgery procedure. The positions of the index markers are used as a reference, so the position can be replicated during future surgical or imaging procedures. This system allows a portion of a patient's body to be targeted for surgery, or for exact positioning to be replicated for repeated imaging. The system of this invention differs from the Prior Art by providing for different means for obtaining the desired outcome of expediting patient positioning within a medical imaging system under a variety of circumstances and conditions, and allowing for the patient to be positioned at the desired imaging point with every imaging procedure, rather than only repeated imaging procedures. By eliminating unnecessary steps through the development of a continuous positioning procedure, this invention should improve positioning accuracy and minimize total procedure time, thus providing better quality images and improved patient comfort.

### SUMMARY OF THE INVENTION

It is an object of this invention to describe a system for the automated localization of a patient in preparation for a medical imaging procedure, such as but not limited to a magnetic resonance imaging (MRI) procedure. Other applicable medical imaging techniques can include positron emission tomography (PET) procedures, optical tomography, single photon emission computed tomography (SPECT), and computerized axial tomography (CAT) procedures.

It is a further object of this invention to describe a system for patient positioning during a medical imaging procedure that operates under a process that provides for essentially continuous positioning feedback as opposed to the step process of Prior Art. After the anatomy of interest has been identified and indicated it is moved to the magnet isocenter in one step, essentially eliminating the extraneous steps currently necessary in the art of centering the anatomy of interest at a superfluous point in space that is a predetermined distance from the isocenter of the imaging system.

It is another object of this invention to describe a system of automated patient localization for medical imaging procedures that will expedite the process of medical imaging, decreasing the time necessary for an imaging session, thus effectively increasing throughput of the imaging center, as well as decreasing the time necessary from the patient to complete the imaging procedure.

It is yet another object of this invention to describe a system of automated patient localization for medical imaging procedures that will decrease the human intervention necessary for positioning and preparing the patient for the scanning procedure, thus eliminating opportunities for human error and increasing the reliability of the imaging procedure.

The invention meets these objects by providing a system consisting of an indicator for identifying the patient anatomy of interest and a controlling unit for advancing the indicated patient anatomy of interest to the desired reference point of the imaging system, for example the isocenter of an MRI system. The indicator may take on many forms, such as but not limited to a fiducial marker, acoustic sensor, touch sensor, special positioning indicator, or a software algorithm. The controlling unit will be such that it will work in conjunction with the type of indicator that is chosen.

These and other objects of the present invention will become more readily apparent from a reading of the following detailed description taken in conjunction with the accompanying drawings wherein like reference numerals indicate similar parts, and with further reference to the appended claims.

### BRIEF DESCRIPTION OF THE DRAWINGS

The invention may take physical form in certain parts and arrangements of parts, numerous embodiments of which will be described in detail in the specification and illustrated in the accompanying drawings that form a part hereof, and wherein:

FIG. 1 is a perspective view of a horizontal field MRI system utilizing the Prior Art system of patient localization;

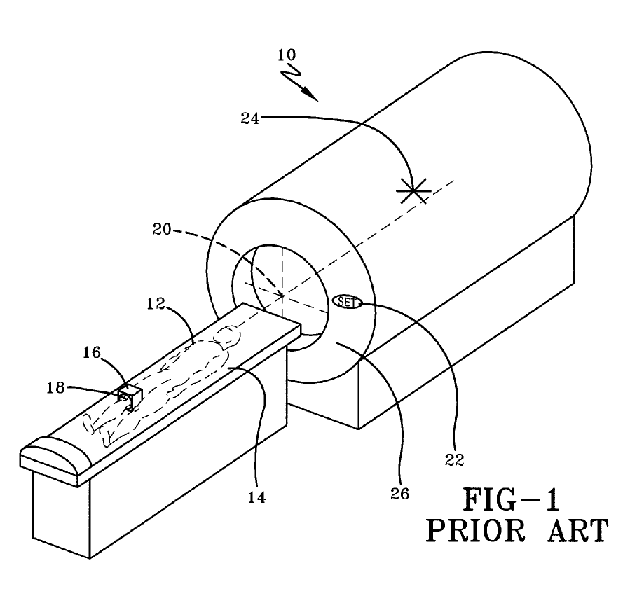

FIG. 2 is a perspective view of a horizontal field MRI system utilizing an embodiment of the invention for patient localization showing the use of a physical indicator and controlling unit;

FIG. 3 is a perspective view of a horizontal field MRI system utilizing another embodiment of the invention showing the use of a positioning wand;

FIG. 4 is a perspective view of a horizontal field MRI system utilizing another embodiment of the invention showing the use of a movable device positioned above the patient;

FIG. 5 is a perspective view of a horizontal field MRI system utilizing a further embodiment of the invention showing the use of a software algorithm for patient localization and positioning;

FIG. 6 is a flow chart illustrating the MR signal method of an embodiment of this invention; and

FIG. 7 is a flow chart illustrating the image identification software method of an embodiment of this invention.

### DETAILED DESCRIPTION OF THE INVENTION

Referring now to the drawings wherein the showings are for purposes of illustrating numerous embodiments of the invention only and not for purposes of limiting the same, the figures illustrate the novel idea of a system for the automated patient localization during medical imaging techniques.

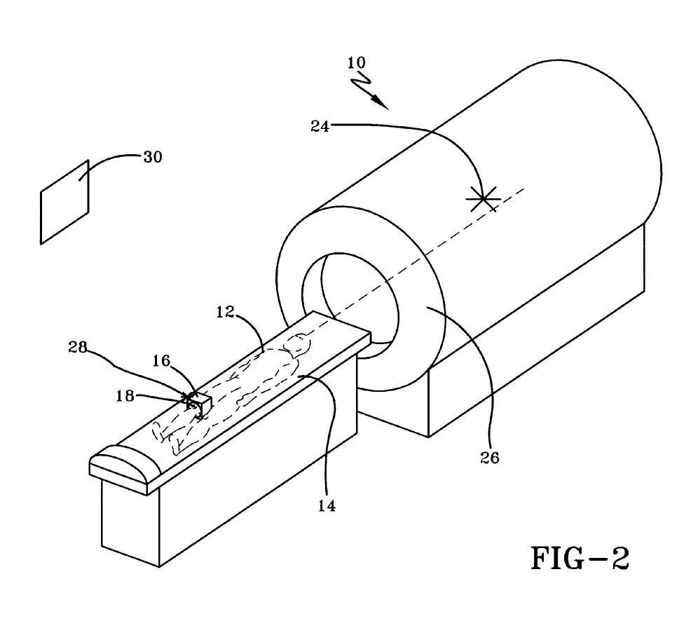

This invention is applicable to any medical imaging system that necessitates patient localization for its proper function, such as magnetic resonance imaging (MRI) systems, positron emission tomography (PET) systems, optical tomography, single photon emission computed tomography (SPECT), and computerized aided tomography (CAT) systems. FIG. 2 illustrates a horizontal field magnetic resonance imaging (MRI) system incorporating a preferred embodiment of the current invention for automated patient positioning. This invention is applicable to any MRI system, and is not limited by the field orientation, field strength, or architecture of the system. The invention applies to, but is not limited to, vertical or horizontal field systems, low, mid or high field systems, open or closed systems, as well as any system that utilizes an arbitrary field strength in an arbitrary direction.

Typically, patient 12 lies on patient table 14 that has been pulled out of MRI system 10 to allow patient 12 to be positioned thereupon. RF Coil 16 is then centered on anatomy of interest 18, which in FIG. 2 is the patient's knee. The RF coil may be positioned around the anatomy of interest, under, or over the target area as the situation dictates to one skilled in the art. Occasionally, the magnet used in some MRI systems are capable of transmitting and receiving, making no RF coil necessary. This invention does not limit the necessity or positioning of the RF coil and can be utilized with any RF coil configuration.

In a preferred embodiment, at least one location is marked with an indicator 28, as being the anatomy of interest 18. It is within the scope of the invention to incorporate the marking of more than one location as the anatomy of interest, as some imaging procedures require multi-station positioning, for example spine or contrast agent imaging procedures. The indicator is such that controlling unit 30 acting as a means of detection senses the initial position of indicator 28 and advances patient table 14 into MRI system 10 such that the anatomy of interest 18 is positioned at isocenter 24 of magnet 26, or any other predefined location within system 10. The invention does not limit the final position of the anatomy of interest to the isocenter of the magnetic field of the system, and could intend for the anatomy of interest to have a final location at any predetermined spot within the MRI system, however, the isocenter is used as the final position throughout the entirety of this description of the invention for brevity and illustrative purposes.

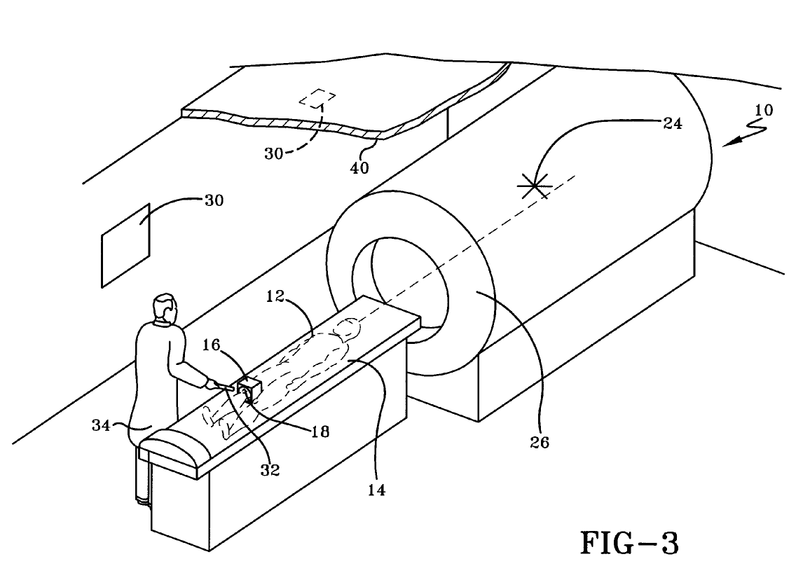

Indicator 28 used to mark anatomy of interest 18 can take on many forms. Some non-limiting examples of indicators utilized in certain embodiments include fiducial markers, light reflecting markers and touch sensitive markers, buttons, or indicators of any sort. A touch sensitive tape or touch sensitive screen can be placed alongside the patient for identification of the anatomy of interest. As shown in FIG. 3, localization wand 32 can be used, wherein technician 34 monitoring the imaging session can activate wand 32 in close proximity to anatomy of interest 18. Wand 32 will produce a visual notification such as a flash of light, a audible notification such as a beep, or another indication of the location of the tip of the wand. Another foreseen indication method (not shown) includes a system of cameras can be utilized to portray an image of the patient onto a touch sensitive screen, allowing the anatomy of interest of the unique patient to be identified by the technician. It is essential the indicator not interfere with the performance or imaging of the medical imaging system. For example, any indicator used with an MRI system must be such that no artifacts or other imaging problems are caused by the material used to construct the indicator or by the method used to secure the indicator in place.

The nature of indicator 28, shown in FIG. 2, is chosen in accord with the method employed by MRI system 10 and controlling unit 30 to monitor the position of indicator 28, and hence anatomy of interest 18. For example, a typical MRI fiducial marker may be used as the indicator if the MRI system's controlling unit is using MRI-based fiducial sensing to locate the indicator. Further examples include the indicator being an optical or acoustic target if the system's controlling unit uses an optical or acoustical tracking system to monitor the location of the indicator.

The at least one indicator 28 may be positioned to mark anatomy of interest 18 in many different ways. Indicator 28 may be placed in a specific relation to the anatomy of interest 18, such as, but not limited to, on top of, below, or a specified distance away from in a specified direction from anatomy of interest 18. In certain embodiments the indicator may be inserted and fixed in its location, unable to slide or be moved to another location without being removed from the first location. In other embodiments the indicator may be movable on a slider, groove, track, or other movable device integrated with, on top of, or into the patient table that may or may not have a locking mechanism to ensure the positioning of the indicator in relation to the anatomy of interest.

Controlling unit 30 is associated with and able to read the signal or communication mode output by the type of indicator 28 being used for the particular imaging system and acts as the detection means of the indicators. For example, with a traditional MRI fiducial, a controlling unit capable of sensing MRI fiducial markers would be necessary. If a localization wand is utilized, as shown in FIG. 3, controlling unit 30, most likely consisting of a series of sensors on the walls and/or ceiling 40 of the room that are capable of identifying the position of the visual, audible, or other notification indicating the tip of the localization wand would be required. Controlling unit 30, of FIG. 2, would preferably be placed within the vicinity of imaging system 10 for uninterrupted and clear reception of indicator 28, and for reliable functioning of the patient localization system.

Controlling unit 30 may monitor indicator 28 by one of a number of different techniques, a non-limiting list including the use of sliding resistors, cable operated potentiometers, linear arrays of mechanical or electrical switches that identify different positions, or linear arrays of optical sensors. Once controlling unit 30 has received the original communication of the initial position of anatomy of interest 18 through a signal or other indication from indicator 28, it will advance patient table 14 from its original position toward reference point 24 within the system. Controlling unit 30 will determine the final position patient table 14 must advance toward by either calculating the distance between the original position of indicator 28 and reference point 24 and moving that set distance, receiving an essentially continual communication from indicator 28 and tracking its advancement toward reference point 24, or by receiving continued signals at a set interval of time or distance to track the advancement of indicator 28 toward reference point 24. The predetermined intervals of time or distance can vary depending on the type of indicator and controlling unit used, as any limitation on time between sampling position will commonly be determined by the limitations of the electronics or mechanics of the indicators and controlling units. The controlling unit may sample the location of the indicators preferably at least once per second, more preferably ten or one hundred times per second, and most preferably continuously.

The reference point, or desired final location of the anatomy, will commonly be the imaging center, or most sensitive location within the imaging system where the highest quality images are taken. The location of reference point 24 indicating the desired final position of anatomy of interest 18 may be pre-programmed in controlling unit 30 or may be identified to controller 30 by an additional indicator 28. If the distance between the original position of indicator 28 and reference point 24 is calculated, that calculation can occur by any means, including but not limited to calculating the angle between controlling unit 30, reference point 24, and current position of indicator 28. Controlling unit 30 will automatically stop patient table 14 when the calculated distance has been traveled by patient table 14 or when indicator 28 in relation to the anatomy of interest 18 has advanced to the same point as reference point 24. The controlling unit should preferably have a high degree of accuracy in calculating the movement of the patient table from the initial location to the desired final location, as a distance as small as a millimeter can affect the images produced by the imaging system. The controlling unit is preferably able to stop the patient table within 2 mm, more preferably within 1 mm, and most preferably less than 1/10 mm from the desired final location.

When more than one indicator is utilized, as in multi-station image sessions, the controlling unit will advance the patient table to the subsequent indicators using the same method as with the first indicator. Occasionally the technician may find it necessary to manually reposition the patient within the system for further imaging positions. If the table is manually repositioned by the technician after the indicated area of interest has reached the desired final position, the controlling unit will return the area of interest to the original desired final position upon the directive of the technician.

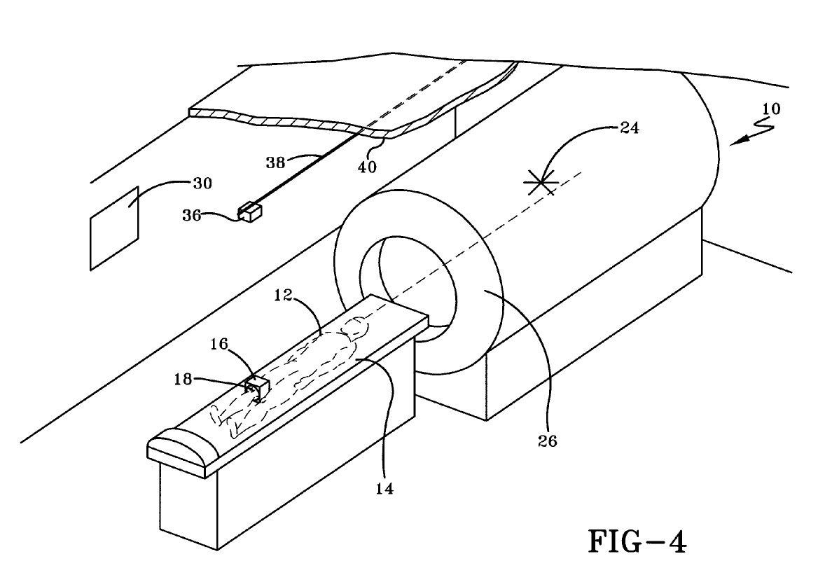

A further aspect of this invention, as shown in FIG. 4, includes movable device 36 affixed to track 38, groove, slider, bar, or other moving means in ceiling 40, or other location above patient 12, that is capable of marking anatomy of interest 18. Movable device 36 will commonly utilize a laser, LED, or other light source that will appear on RF Coil 16 or patients' anatomy of interest 18 when centered correctly. Device 36 may be easily maneuvered to center the marking source, whether it be light or otherwise, on anatomy of interest 18. Once device 36 is properly positioned, controlling unit 30 will sense the position of device 36, and advance patient table 14 into MRI system 10, such that the anatomy of interest 18 is positioned at the isocenter 24 of the system.

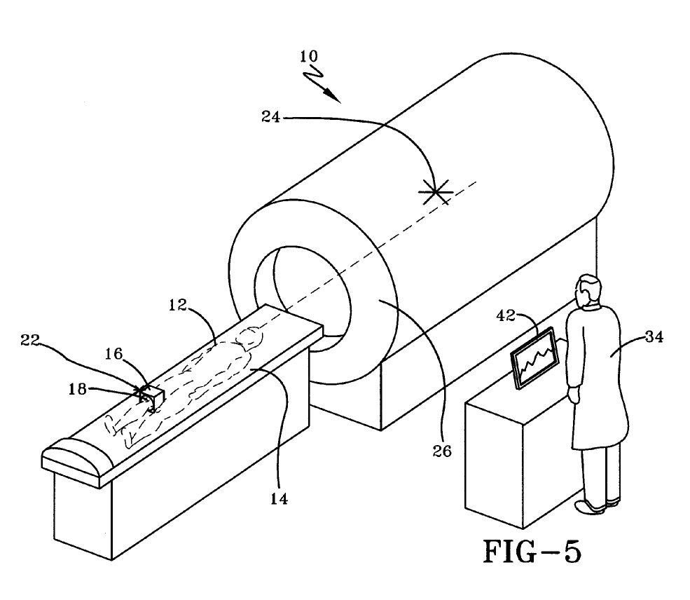

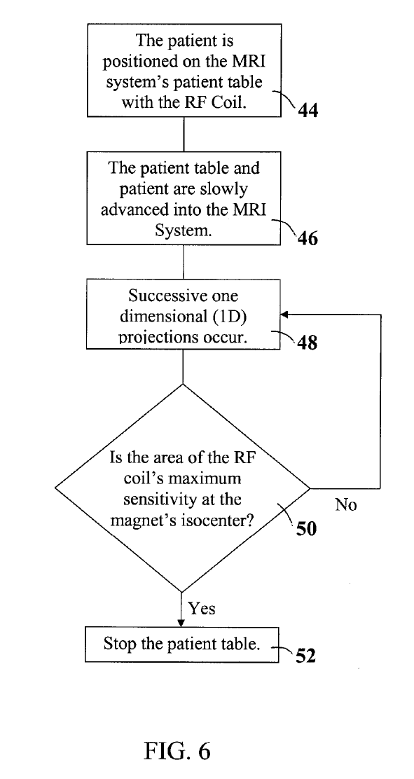

Yet another embodiment of this invention, shown in FIG. 5, entails the use of a software algorithm utilized for patient localization and positioning. A flowchart illustrating the function of this method appears as FIG. 6. One of the software algorithm based methods is a MR signal based method for use with an MRI system. It is known in the art that some RF coils have a characteristic response function with maximum sensitivity occurring in the central region of the coil. Knowing the maximum sensitivity occurs at the center of RF coil 16, as shown in FIG. 5, and also knowing that anatomy of interest 18 is centered in RF coil 16 (corresponding to step 44 in the flowchart of FIG. 6), a software algorithm can run on computer 42 that acts as the image viewer and controls the system software such that the system will sense when the most sensitive region of RF coil 16 is approaching the most sensitive region of the system i.e., isocenter 24, by use of a communication-feedback loop. As patient table 14 slowly advances into the MRI system (step 46 of the flowchart of FIG. 6) fast, successive one-dimensional (1D) projections are collected (step 48 of the flowchart of FIG. 6). The software algorithm is designed to detect the varying sensitivity of the coil by the 1D projections as the center of RF coil 16 approaches isocenter 24 of the system (step 50 of the flowchart of FIG. 6). The software algorithm, which is controlling the movement of patient table 14 while simultaneously sensing the increasing sensitivity of RF coil 16 response, stops patient table 14 as the RF coil's region of maximum sensitivity meets magnet's 26 region of maximum sensitivity, putting the anatomy of interest 18 directly at isocenter 24 (step 52 of the flowchart of FIG. 6). Technician 34 or other professional monitoring the imaging session may run a localization scan to fine tune the correct couch position.

Thus, the system is essentially using the sensitivity of RF coil 16 as the indicator, and a software algorithm as the controlling unit to recognize the sensitivity of RF coil 16 from a series of quickly taken scans as the patient 12 is slowly advanced into the MRI system. When the maximum sensitivity of RF coil 16, and thus the anatomy of interest 18 is recognized by the software controlling unit, patient table 14 will be advanced to the correct position such that the anatomy of interest 18 is centered at isocenter 24 of the system. However, this method of patient localization may have less accuracy than the aforementioned methods utilizing a physical indicator and controlling unit system. Nevertheless, the accuracy of the MR signal method would be acceptable for most types of imaging studies. Pilot scans, locator scans, or other fast scans for the purpose of quickly locating the position of the body in the system would be utilized to fine tune the exact position that images should be taken.

The MR signal method for patient localization may not be appropriate in all situations, as it has inherent limitations, such as use with phased array coils. These are coils with elements that combine differently than a single element, creating numerous areas of high sensitivity within the RF coil rather than having just one area of maximum sensitivity at the center of the coil. The software algorithm will no longer be able to operate under the principle that the anatomy of interest is located at the point of highest sensitivity within the coil as there are now numerous areas of high sensitivity, not all of which are centered on the anatomy of interest. Applications that utilize only certain elements of the phased array RF coil may still be able to use this embodiment of the invention, as there would be a limited number of areas of high sensitivity, most of which would be near or on the anatomy of interest.

Complications will also arise when whole body studies, run-off studies, or other imaging protocols are used that require the patient table to move during the scan protocol. In this case, the MR signal method of the invention could still be utilized, but would not be as efficient as in other situations. The patient could be advanced into the system as the software algorithm searches for the approaching optimal sensitivity of coil 16, of FIG. 5, to reach isocenter 24 of magnet 26. Patient table 14 can be stopped at isocenter 24, at which time the moving table imaging study can begin, with each set of elements being switched on or off as the patient passes through isocenter 24 of magnet 26, allowing the software algorithm to find the optimal response location for each stage of the study. When the moving table portion of the study is complete, the patient will be returned to the position of the original scan (i.e. isocenter).

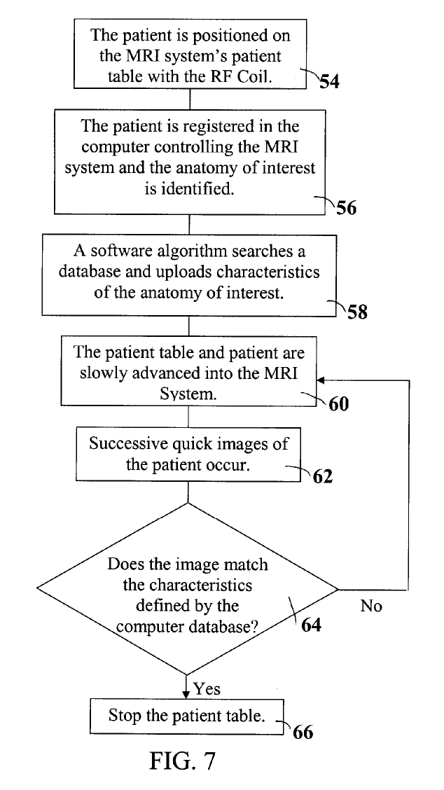

A further aspect of the invention (shown in the flowchart FIG. 7) encompasses another software based method of identification and positioning of the anatomy of interest. The technologist positions the patient on the patient table of the MRI System (step 54 of FIG. 7). Next, the technologist who runs the imaging procedure registers the patient in the computer controlling the MRI system, or other type of medical imaging system (step 56 of FIG. 7). As the patient is registered, the anatomy of interest is identified and linked to the electronic folder for image storage created for the patient. A software algorithm can search a database of characteristics had by the anatomy of interest identified in the patient registration information, such as but not limited to approximate size in comparison to surrounding structures, approximate distance in comparison to surrounding structures, or approximate shape or tissue structure (step 58 of FIG. 7). The patient is slowly advanced into the imaging system (step 60 of FIG. 7) as quick images of the patient are taken (step 62 of FIG. 7). The software algorithm compares the images with the characteristics of the anatomy of interest (step 64 of FIG. 7). When the characteristics are recognized, the patient will be stopped so that the anatomy of interest is centered at the isocenter of the magnet, or other reference point within the imaging system (step 66 of FIG. 7). This embodiment of the invention uses the anatomy of interest as the indicator, and the software algorithm as the controlling unit.

For example, if the liver is the anatomy of interest of a certain patient, the patient is registered in the system, and the liver is identified on the patient's electronic profile. The patient is placed on the MRI system patient table and a torso coil (a type of RF coil) is placed around the patient's midsection. Once activated, the software algorithm instantaneously accesses its database and finds that the characteristics of the liver include that it is a predetermined distance from a certain sized black region associated with the lung. The software algorithm slowly advances the patient table with the patient on it, into the MRI system, as it searches for the sudden signal void and black region associated with the lung, and continues to advance for a predetermined distance after signal returns, indicating the lung has been passed.

The anatomy of interest identification embodiment previously described can be used by itself on any medical imaging system, or in combination with the previously mentioned MR Signal method for patient localization on MRI systems. For example, the MR Signal method can be utilized, and then the anatomy of interest identification method can be used as a secondary check to ensure the proper location was identified and centered at isocenter of the magnet, or the anatomy of interest identification method can be used and the MR Signal method can be used as the secondary assurance.

Described above is a process for patient localization in a medical imaging system that comprises of orienting the patient on the patient table and with respect to any additional necessary hardware, providing an indication of the patient anatomy of interest, and advancing the patient and the RF coil to a reference point within the medical imaging system in response to the indication, as well as a system for use with the process. The process and system can be used on any medical imaging system, such as an MRI system, PET system, and CAT system. The indication of the patient anatomy of interest and the controlling unit can take on many forms, and utilize electrical, acoustical, light sensitive, optical, touch sensitive, MR signals and other methods for proper operation.

In the foregoing description, certain terms have been used for brevity, clearness, illustration and understanding; but no unnecessary limitations are to be implied therefrom beyond the requirements of the prior art, because such terms are used for descriptive purposes and are intended to be broadly construed. Moreover, this invention has been described in detail with reference to specific embodiments thereof, including the respective best modes for carrying out each embodiment. It shall be understood that these illustrations are by way of example and not by way of limitation.

What is claimed is:

1. A process for patient localization within a medical imaging system, said system having a first and second signal means for identifying patient position which comprises:
(a) manually positioning a patient on a patient table on a track when said table is in an initial position outside of said system;
(b) manually positioning said first signal means adjacent an area of interest on said patient in said initial position and
(i) said first signal means communicating said initial patient position to a detection means and essentially continuously communicating subsequent patient positions;
(ii) said second signal means communicating a desired final patient position location to said detection means;
(c) essentially continuously monitoring and comparing said initial and subsequent positions to said final position by said detection means and causing said patient to move from said initial position to said final position when said positions are not essentially the same.

2. The process of claim 1 wherein
said final position is at the imaging center of said system.

3. The process of claim 2 wherein
said first signal means is selected from the group consisting of a fiducial marker, a touch sensitive marker, a light sensitive marker, an acoustic sensitive marker, a sliding marker, a movable overhead laser cross-hair, position indicating wand, and a touch sensitive strip.

4. The process of claim 3 wherein
said detection means corresponds in function and operates in conjunction with the chosen said first signal means.

5. The process of claim 3 wherein
said medical imaging system is selected from the group consisting of a magnetic resonance imaging system, a computer aided tomography system, a positron emission tomography system, an optical tomography system, and a single photon emission computed tomography system.

6. The process of claim 1 which further comprises
returning said area of interest to said final position after subsequent repositioning of said area of interest during the imaging session.

7. A process for patient localization within a medical imaging system, said system having a first and second signal means for identifying patient position which comprises:
(d) manually positioning a patient on a patient table on a track when said table is in an initial position outside of said system;
(e) manually positioning said first signal means adjacent an area of interest on said patient in said initial position and
(i) said first signal means communicating said initial patient position to a detection means;
(ii) said second signal means communicating a desired final patient position location to said detection means;
(f) comparing said initial to said final position by said detection means, determining a distance between said initial and said final position, and causing said patient to move said distance without stopping at any point before said final position.

8. The process of claim 7 wherein
said final position is at the imaging center of said system.

9. The process of claim 8 wherein
said first signal means is selected from the group consisting of a fiducial marker, a touch sensitive marker, a light sensitive marker, an acoustic sensitive marker, a sliding marker, a movable overhead laser cross-hair, and a touch sensitive strip.

10. The process of claim 9 wherein
said detection means corresponds in function and operates in conjunction with the chosen said first signal means.

11. The process of claim 10 wherein
said medical imaging system is selected from the group consisting of a magnetic resonance imaging system, a computer aided tomography system and a positron emission tomography system.

12. The process of claim 7 which further comprises
returning said area of interest to said final position after subsequent repositioning of said area of interest during the imaging session.

13. A process for patient localization within a magnetic resonance imaging system, said system using the location of strongest MR signal from an RF coil with respect to isocenter of the magnetic field of said system for identifying patient position which comprises:
(g) manually positioning a patient on a patient table on a track when said table is in an initial position outside of said system;
(h) manually positioning said RF coil adjacent an area of interest on said patient in said initial position and
(i) said strength of MR signals from said RF coil communicating said initial patient position to a detection means and essentially continuously communicating subsequent patient positions;
(ii) said sensitivity of said isocenter of said magnetic field of said system communicating a desired final patient position location to said detection means;
(i) essentially continuously monitoring and comparing said initial and subsequent positions to said final position by said detection means and causing said patient to move from said initial position to said final position when said positions are not essentially the same.

14. The process of claim 13 which further comprises
returning said area of interest to said final position after subsequent repositioning of said area of interest during the imaging session.

15. A process for patient localization within a magnetic resonance imaging system, said system using an indication of an area of interest on said patient and an indication of said area of interest from a database for identifying patient position which comprises:
(j) manually positioning a patient on a patient table on a track when said table is in an initial position outside of said system;
(k) manually indicating said area of interest on said patient's electronic profile;
(l) collecting MR images at predefined increments of time and
(i) said images communicating said initial patient position to a detection means and essentially continuously communicating subsequent patient positions;
(ii) characteristics of said area of interest from a database linked to said patient electronic profile communicating a desired final patient position location to said detection means;
(m) essentially continuously monitoring and comparing said initial and subsequent positions to said final position by said detection means and causing said patient to move from said initial position to said final position when said positions are not essentially the same.

16. The process of claim 15 which further comprises
returning said area of interest to said final position after subsequent repositioning of said area of interest during the imaging session.
